(19) United States
(12) Patent Application Publication
(10) Pub. No.: US 2017/0028196 A1
(43) Pub. Date: Feb. 2, 2017

(54) THERAPEUTIC STIMULATION SYSTEM

(71) Applicant: JAHN JEFFERY STOPPERAN, Lakeville, MN (US)

(72) Inventor: JAHN JEFFERY STOPPERAN, Lakeville, MN (US)

(21) Appl. No.: 14/996,058

(22) Filed: Jan. 14, 2016

Related U.S. Application Data
(60) Provisional application No. 62/282,097, filed on Jul. 27, 2015.

(51) Int. Cl. A61N 1/36; A61F 7/00; A61N 1/08

(52) U.S. Cl. CPC A61N 1/36021; A61N 1/08; A61F 7/007; A61F 2007/0071; A61F 2007/0093

(57) ABSTRACT

A therapeutic stimulation system includes at least one pad assembly and a control device for electrically generating therapeutic stimulation. The pad assembly includes a heating layer and a pad identification circuit. The heating layer includes a low-wattage resistive heating element. The pad identification circuit includes a storage device that can store information identifying the pad assembly. The control device operates to identify the pad assembly based on the information and determine an operational scheme specific to the pad assembly.

### CROSS-REFERENCE TO RELATED APPLICATIONS

The present application claims priority from U.S. Provisional Patent Application No. 62/282,097, filed on Jul. 27, 2015, the disclosure of which is hereby incorporated by reference in its entirety.

### BACKGROUND

Healthcare industries, such as chiropractic and therapeutic treatments, provide services for pain relief resulting from various sources including physical injury, aging, and disease. In addition to mechanical methods such as realignment, stretching, extension, and even acupuncture, skin-applied treatments are also used.

One of such skin-applied treatments is to use a stimulation pad that provides localized therapeutic stimulation. One example of the stimulation pad produces transcutaneous electrical nerve stimulation (TENS) to relieve chronic pain and produce muscle building stimulation. Such electrical stimulation can also treat injured and weakened soft body tissue.

### SUMMARY

In general terms, this disclosure is directed to a therapeutic stimulation system. In one possible configuration and by non-limiting example, the system includes a pad assembly including a pad identification circuit configured to identify the pad assembly communicatively connected to a control device. The pad assembly can include a heating layer as well, including a low-wattage resistive heating element. Various aspects are described in this disclosure, which include, but are not limited to, the following aspects.

One aspect is a system for electrically generating therapeutic stimulation. The system includes at least one pad assembly and a control device. Each pad assembly may include a skin attachment surface, a heating layer, and a pad identification circuit. The skin attachment surface includes an adhesive formed thereon. The adhesive provides detachable, reusable adhesion to a skin area of a patient. The heating layer is arranged within the pad assembly and includes a low-wattage resistive heating element. The pad identification circuit includes a storage device that can store information identifying the pad assembly. The control device is communicatively connected to the pad identification circuit and electrically connected to the heating layer. The control device is configured to identify the pad assembly based on the information; and determine an operational scheme specific to the pad assembly. In certain examples, the storage device may store information describing one or more physical characteristics of the pad assembly.

In certain examples, the pad assembly may further include one or more electrical stimulation circuits including a plurality of electrical stimulation electrodes spaced apart from each other.

In certain examples, the operational scheme includes at least one of electric power delivered to the heating layer of the pad assembly and a stimulation pattern applied via the plurality of electrical stimulation electrodes.

In certain examples, the control device is configured to monitor a first cumulative time that the pad has been in use and generate a pad replacement notification based on the first cumulative time exceeding a predetermined pad service time. The pad replacement notification is adapted to indicate that the pad assembly should be replaced.

In certain examples, the control device is further configured to generate a pad replacement reminder notification when the first cumulative time exceeds a first threshold.

In certain examples, the skin attachment surface is removable from the pad assembly and replaceable. Further, the physical characteristics of the pad assembly may include physical characteristics of the skin attachment surface. The control device may monitor a second cumulative time that the skin attachment surface has been used and generate a layer replacement notification when the second cumulative time exceeds a layer service time. The layer replacement notification is adapted to inform that the skin attachment surface needs to be replaced. The control device may be further configured to generate a layer replacement reminder notification when the second cumulative time exceeds a second threshold. In certain examples, the pad identification device is incorporated in the skin attachment surface.

In certain examples, the heating layer includes a plurality of heating zones controlled independently based on the operational scheme. In certain examples, the plurality of electrical stimulation electrodes defines a plurality of electrical stimulation zones controlled independently based on the operational scheme.

Another aspect is a pad assembly for electrically generating therapeutic stimulation. The pad assembly includes a skin attachment surface, a heating layer, and a pad identification circuit. The skin attachment surface includes an adhesive formed thereon, which provides detachable, reusable adhesion to a skin area of a patient. The heating layer is arranged within the pad assembly and includes a low-wattage resistive heating element. The pad identification circuit includes a storage device that can store information identifying the pad assembly. The pad identification circuit is configured to be communicatively connected to a control device and transmit the information to the control device to enable the control device to identify the pad assembly and determine an operational scheme specific to the pad assembly.

In certain examples, the heating layer includes a plurality of heating zones controlled independently based on the operational scheme. In certain examples, the pad assembly may further include one or more electrical stimulation circuits including a plurality of electrical stimulation electrodes spaced apart from each other and attachable to the skin area of the patient.

In certain examples, the operational scheme includes at least one of electric power delivered to the heating layer of the pad assembly and a stimulation pattern applied via the plurality of electrical stimulation electrodes.

In certain examples, the skin attachment surface is removable from the pad assembly and replaceable. The physical characteristics of the pad assembly include physical characteristics of the skin attachment surface, and the pad identification device is incorporated in the skin attachment surface.

In certain examples, the plurality of electrical stimulation electrodes defines a plurality of electrical stimulation zones controlled independently based on the operational scheme.

The above features and advantages and other features and advantages of the present teachings are readily apparent from the following detailed description for carrying out the present teachings when taken in connection with the accompanying drawings.

### BRIEF DESCRIPTION OF THE DRAWINGS

FIG. 1 schematically illustrates a system for electrically generating therapeutic stimulation in accordance with an exemplary embodiment of the present disclosure.

FIG. 2 is a schematic block diagram of the system of FIG. 1.

FIG. 3 is a schematic block diagram of a control device of the system of FIG. 1.

FIG. 4 is a schematic perspective view of an example pad assembly of the system.

FIG. 5 is a schematic perspective view of a plurality of pad assemblies of the system.

FIG. 6 is a schematic perspective view of another example pad assembly of the system.

FIG. 7 is a schematic expanded view of the pad assembly of FIG. 4.

FIG. 8 is a schematic cross sectional view of the pad assembly of FIG. 4.

FIG. 9 illustrates an example heating layer of the pad assembly.

FIG. 10 schematically illustrates other examples of the heating layer.

FIG. 11A is a schematic view of an example pad identification device of the pad assembly.

FIG. 11B is a schematic view of another example pad identification device of the pad assembly.

FIG. 12 illustrates various example shapes of the pad assembly.

FIG. 13 is a flowchart illustrating an example method of operating the system of FIG. 1.

FIG. 14 is an example of pad control scheme data.

FIG. 15 is a schematic expanded view of another example pad assembly.

FIG. 16 is a flowchart illustrating an example method of operating the system with the pad assembly of FIG. 15.

FIG. 17 is an example of adhesive layer data.

FIG. 18 schematically illustrates a heating layer including a plurality of heating zones.

FIG. 19 illustrates an exemplary architecture of a control device of the system of FIG. 1.

### DETAILED DESCRIPTION

Various embodiments will be described in detail with reference to the drawings, wherein like reference numerals represent like parts and assemblies throughout the several views.

In general, a therapeutic stimulation system includes at least one pad assembly and a control device for electrically generating therapeutic stimulation. The pad assembly provides localized stimulation using heat to activate tissue blood flow and muscle action, thereby accelerating healing and pain relief. The pad assembly includes a heating layer and a pad identification circuit. The heating layer includes a low-wattage resistive heating element. The pad identification circuit includes a storage device that can store information identifying the pad assembly. The control device operates to identify the pad assembly based on the information and determine an operational scheme specific to the pad assembly. In certain examples, the pad assembly is further configured to electrical stimulation, such as TENS-type soft body tissue stimulation. The pad assembly is controlled to generate a variety of different electrical and/or thermal stimulation patterns of a skin area.

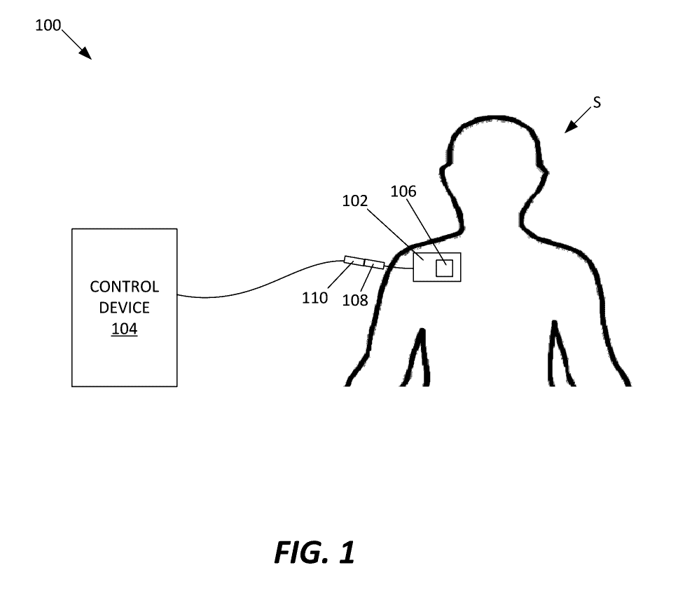

FIG. 1 schematically illustrates an example system 100 for electrically generating therapeutic stimulation for a subject S. The system 100 includes a pad assembly 102 and a control device 104.

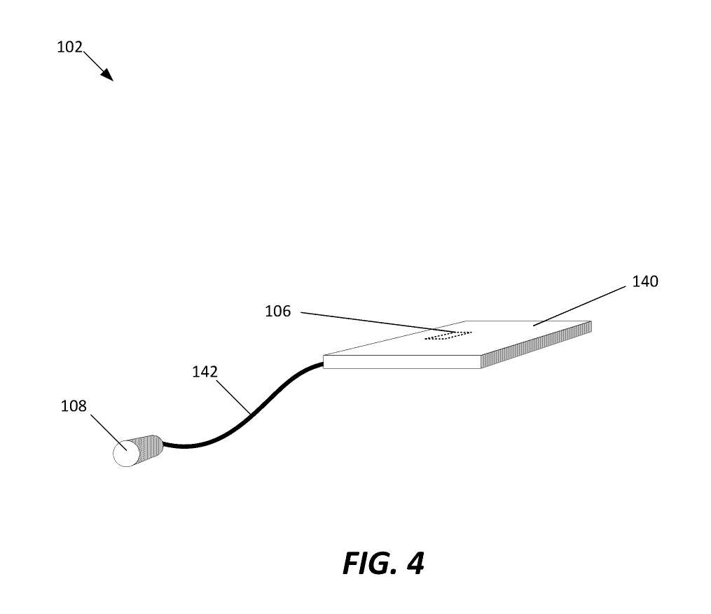

The pad assembly 102 is configured to be detachably attached to a portion of the subject's body surface or the subject's skin area. The pad assembly 102 can be worn on different locations of the subject body for different therapeutic purposes. The pad assembly 102 includes a pad identification device 106 adapted to enable the control device 104 to identify the pad assembly 102 associated with the control device 104. As described herein, the pad identification device 106 at least partially defines a pad identification circuit and is also referred to herein as a pad identification circuit. In some examples, the pad assembly 102 is configured to provide a combination of electrical stimulation, such as TENS-type soft body tissue stimulation, and thermal stimulation of a skin area or body tissue. An example of the pad identification device 106 (and the pad identification circuit) is described in more detail with respect to FIG. 4.

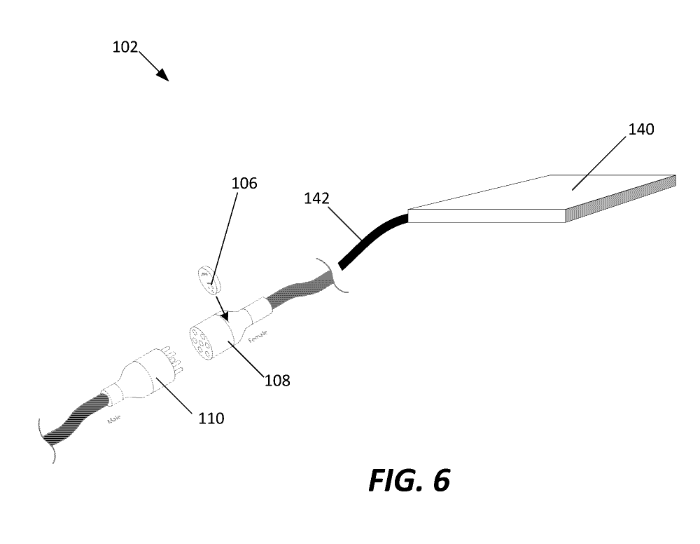

The control device 104 is communicatively connectable to the pad assembly 102. The control device 104 can be coupled to the pad assembly 102 in various manners. The control device 104 controls the pad assembly 102 to generate various patterns and profiles of electrical and/or heating stimulation to an isolated area of the body on which the pad is attached. In some examples, the pad assembly 102 can be connected to the control device 104 via a first connector 108 of the pad assembly 102 and a second connector 110 of the control device 104. In some examples, the first and second connectors 108 and 110 are configured to be complementarily coupled to each other. For examples, as illustrated in FIG. 6, the first connector 108 includes one or more female sockets configured to receive one or more corresponding male pins extending from the second connector 110.

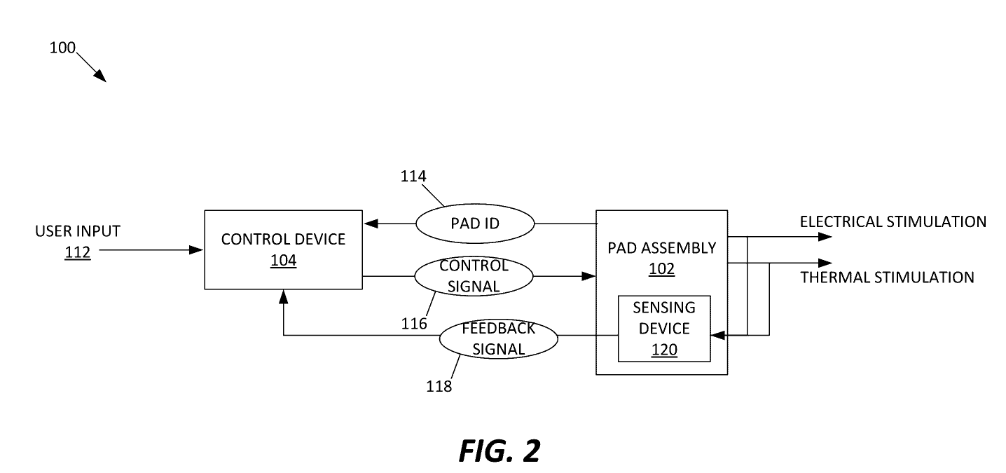

Referring to FIG. 2, the control device 104 operates to control the pad assembly 102 based on a user input 112. As described below, the control device 104 includes a user interface to receive the user input. In some examples, the user input includes a switch on/off, a heating temperature setting, an electrical stimulation setting, a timer, and any other inputs associated with the operation of the pad assembly 102.

As described herein, the pad assembly 102 (e.g., the pad identification device 106) stores information 114 identifying the pad assembly 102, and transmits this pad ID information 114 to the control device 104 when the pad assembly 102 is connected to the control device 104. Based on the user input 112 and/or the pad ID information 114, the control device 104 generates and transmits a control signal 116 to the pad assembly 102 to control the pad assembly 102. The pad assembly 102 can selectively generate electrical stimulation and thermal stimulation (i.e. heating), or generate a combination of electrical and thermal stimulations, in various manners (e.g., various patterns or profiles of heating and/or electrical stimulation) in accordance with the control signal 116.

In some examples, the system 100 employs a closed loop control scheme that monitors the output of the pad assembly 102, such as the thermal output pattern and/or the electrical stimulation output pattern, and transmits the output signal 118 back to the control device 104. The control device 104 can compare the output signal with the desired output to reduce an error between the actual and desired outputs, thereby improving the control of the pad assembly 102 and providing precise thermal management. Alternatively, in other embodiments, the system 100 can use other control schemes, such as an open loop control.

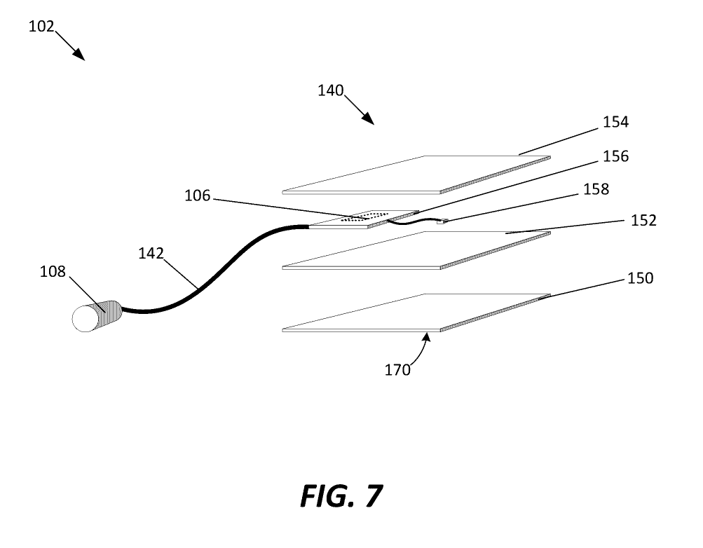

The system 100 can include a sensing device 120 for detecting the output of the pad assembly 102. In some examples, the sensing device 120 is embedded within the pad assembly 102 and configured to monitor one or more parameters, such as temperature and/or electrical signals (e.g., voltage or current), generated by the pad assembly 102. An example of the sensing device 120 is described in more detail with reference to FIG. 7.

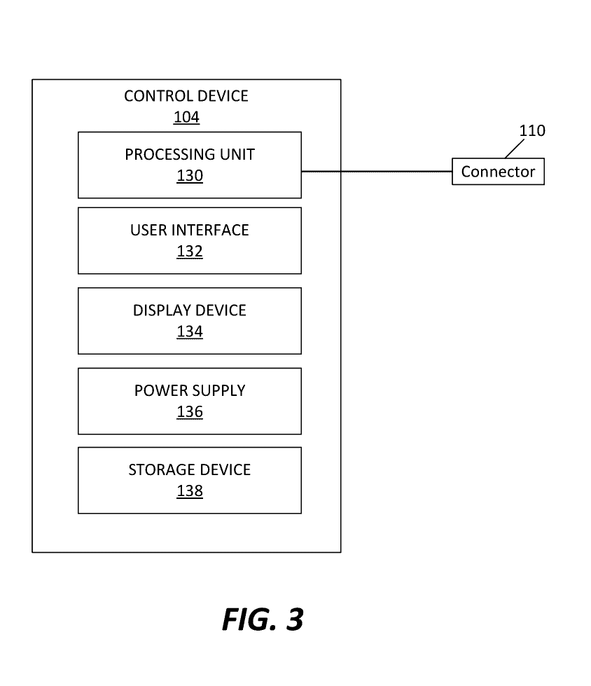

Referring to FIG. 3, the control device 104 includes a processing unit 130 that is used to control the pad assembly 102 by transmitting the control signal 116 to the pad assembly 102. The processing unit 130 can include a microprocessor. The control device 104 further includes a user interface 132 to receive a user input 112, and a display device 134 to display various pieces of information, such as the user input, the identification of the pad assembly 102, the physical characteristics of the pad assembly 102, and the operational status of the pad assembly 102. The control device 104 includes a power supply 136 for providing electric power to electronic components of the pad assembly 102. The power supply 136 can also be used to transmit power to the pad assembly 102 via the control signal 116. In some examples, the power supply 136 includes a battery housed in the control device 104. The battery can be replaceable and/or rechargeable. Alternatively, the battery is external to the control device 104, and portable and connectable to the control device 104. In other examples, the power supply 136 is arranged external to the control device 104, such as an electrical outlet into which the control device 104 is plugged. In yet other embodiments, the pad assembly 102 includes its own power source.

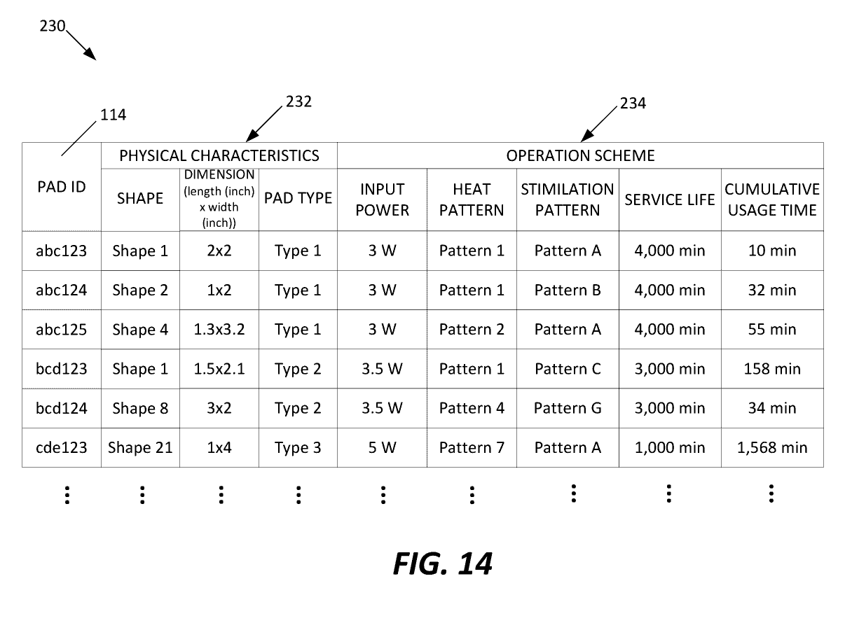

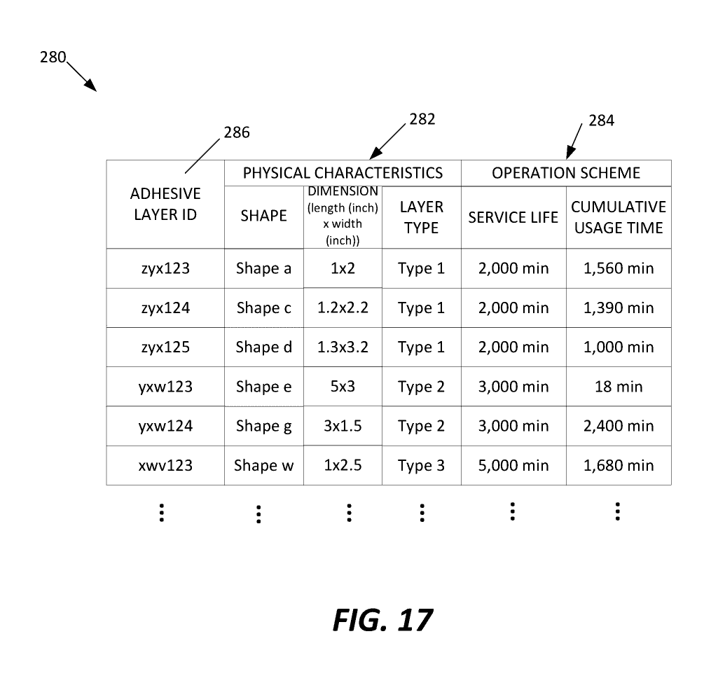

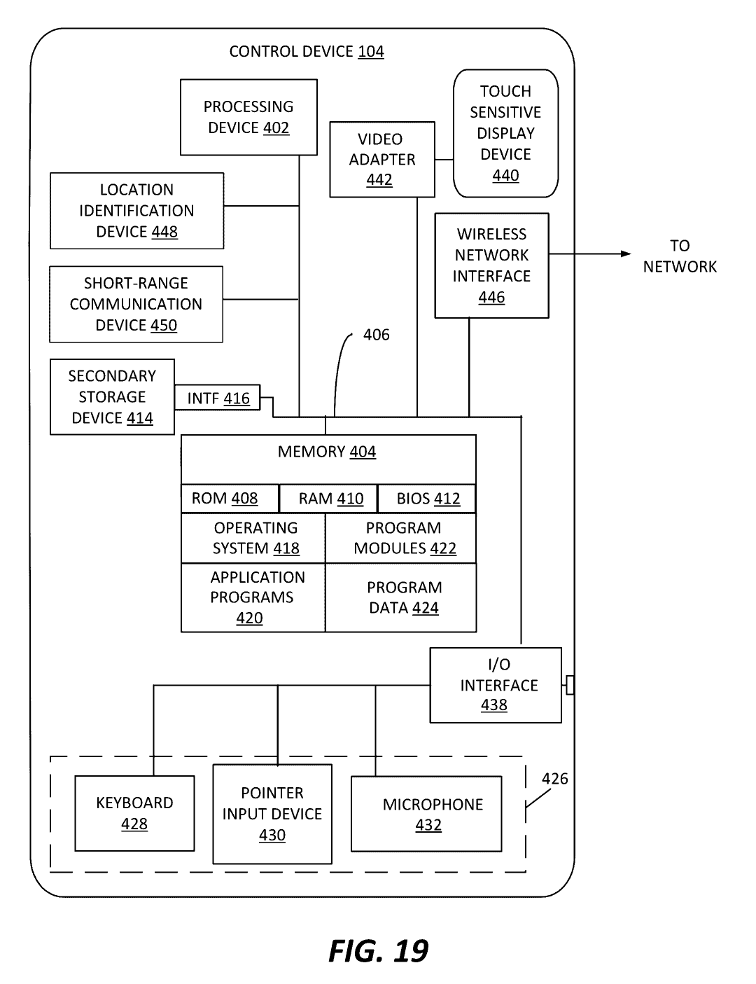

The control device 104 further includes a storage device 138. The storage device 138 can be configured similarly to a memory 404 or a second storage device 414 as shown in FIG. 19. As described herein, the storage device 138 is used to store information about one or more pad assemblies 102 as described with reference to FIGS. 14 and 17.

In some examples, the control device 104 includes a portable device, which can be configured as an independent handheld device or a device that is connected to a movable station or equipment. The control device 104 can also be implemented with various consumer computing devices including a mobile computing device, such as a smartphone, (e.g., an iPhone, an Android operating phone, a Blackberry, a Window operating phone, etc.); a tablet computer (e.g., an iPad), and a personal digital assistant (PDA). The control device 104 can also include a desktop computer, a laptop computer, and/or any other suitable devices operable to send and receive signals, store and retrieve data, and/or execute modules.

In some embodiments, the control device 104 is wirelessly connectable to the pad assembly 102 to control the pad assembly 102. For examples, the pad assembly 102 includes a power source (e.g., a battery) to supply power to the heating element and/or the electrical stimulation element therein, and the control device 104 transmits a radio frequency signal (i.e., the control signal 116) to the pad assembly 102 to control the power supply within the pad assembly 102.

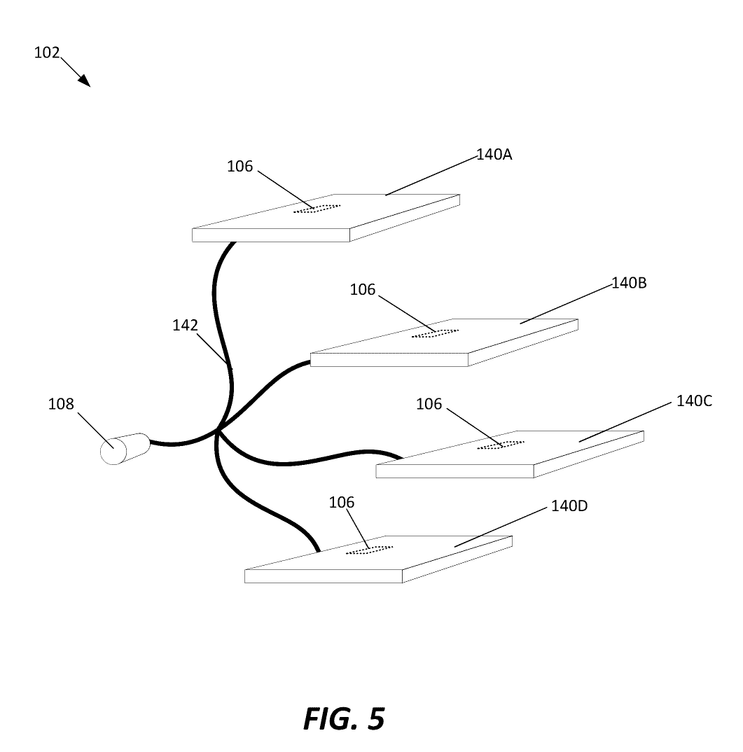

With reference to FIGS. 4 and 5, the pad assembly 102 includes a pad 140 that includes the pad identification device 106. The pad assembly 102 includes a cable 142 extending between the pad 140 and the first connector 108. In some examples, the pad assembly 102 includes a single pad 140, as illustrated in FIG. 4. In other examples, the pad assembly 102 includes a plurality of pads 140 (including 140A, 140B, 140C, and 140D), as illustrated in FIG. 5.

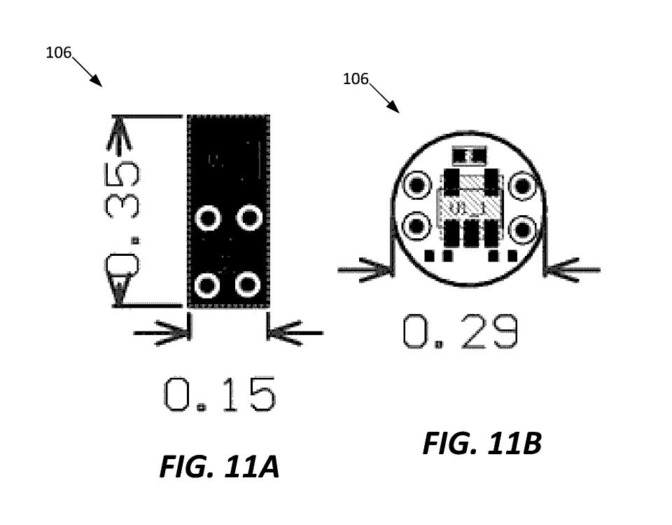

The pad identification device 106 (i.e., the pad identification circuit) operates to store information associated with the associated pad assembly 102. Such information is used to identify one or more pad assemblies 102 connected to the control device 104 and control the pad assemblies 102 as desired. The pad identification device 106 can be arranged in various locations. In some examples, the pad identification device 106 is embedded in the pad 140, as illustrated in FIGS. 4 and 5. Although it is illustrated in FIG. 5 that each of the plurality of pads 140 includes a pad identification device 106, it is also possible to provide only one pad identification device for some or all of the pads 140, or to provide a plurality of pad identification devices for some or all of the pads 140. In other examples, the pad identification device 106 is incorporated in the first connector 108, as shown in FIG. 6. Other locations of the pad identification device 106 are also possible in other embodiments. An example configuration of the pad identification device 106 is illustrated in FIGS. 11A and 11B.

Where the pad assembly 102 includes a plurality of pads 140, each pad 140 can be controlled independently by the control device 104. For examples, the pads 140 can be operated differently, depending on the identification of each pad as described herein. In other examples, the plurality of pads 140 can be controlled identically by the control device 104.

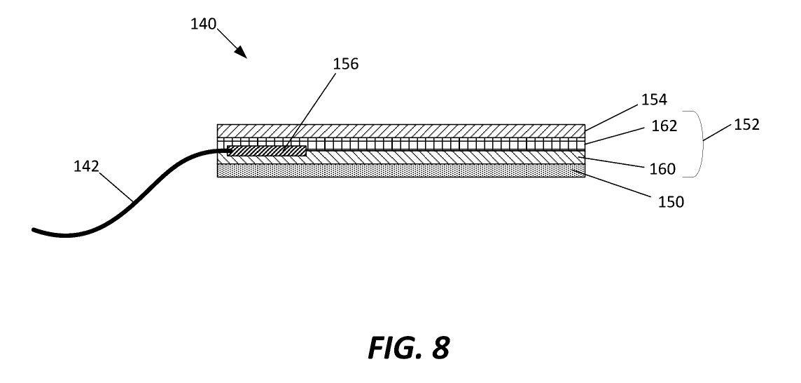

Referring to FIGS. 7 and 8, the pad 140 includes a plurality of layers. In some examples, the pad 140 includes an adhesive layer 150, a stimulation layer 152, and a cover layer 154. Further, a pad circuit 156 is incorporated within the pad 140.

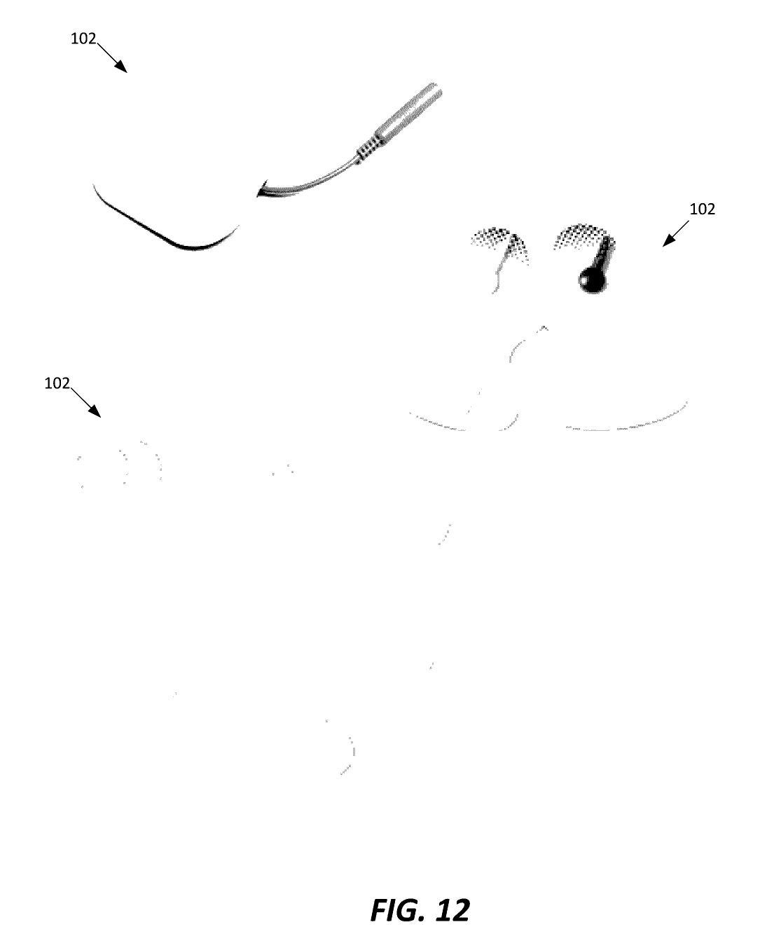

The pad 140 is designed to be flexible and thin enough to follow the contour of a skin surface and to remain in good contact with the surface. In some embodiments, the pad 140 has an overall thickness of about 0.08-0.10 inches. The pad 140 is configured in various shapes, such as square, rectangular, circular, oval, and any other shapes suitable for therapeutic stimulation. The pad 140 can also vary in size for different purposes. Some examples of different shapes and/or sizes of the pad 140 are illustrated in FIG. 12.

The adhesive layer 150 provides a skin attachment surface 170 that includes an adhesive formed thereon. In this document, therefore, the adhesive layer 150 can also be referred to as the skin attachment surface. The adhesive provides detachable, reusable adhesion to a skin area of the subject. In some examples, the adhesive layer 150 is at least partially made of hydrogel. For example, the adhesive layer 150 includes a hydrogel layer, or the skin attachment surface 170 is coated with hydrogels. An example of hydrogel suitable for the adhesive layer of the present disclosure is manufactured by R&D Medical Products of Lake Forest, Calif.

The stimulation layer 152 is designed to provide thermal stimulation (i.e., heating) and/or electrical stimulation of a skin area or body tissue. Such electrical stimulation includes TENS-type soft body tissue stimulation. In some examples, the stimulation layer 152 is operated to provide thermal stimulation only. In other examples, the stimulation layer 152 is operated to provide electrical stimulation only. In yet other examples, the stimulation layer 152 is controlled to provide both thermal and electrical stimulation in various manners. The stimulation layer 152 can be controlled by the control device 104 to selectively generate either or both of electrical thermal stimulations. In some examples, the stimulation layer 152 is activated by the control device 104 based on identification of the pad assembly.

As illustrated in FIG. 8, where the stimulation layer 152 provides a combination of thermal and electrical stimulations, the stimulation layer 152 can have a plurality of layers, including a heating layer 160 and an electrical stimulation layer 162.

The heating layer 160 is configured to provide suitable heating to one or more isolated portions of the subject's body to provide pain relief and body healing. The heat generated by the heating layer 160 is isolated to the specific area on which the pad 140 is attached and thus provides effective therapeutic treatment on the area.

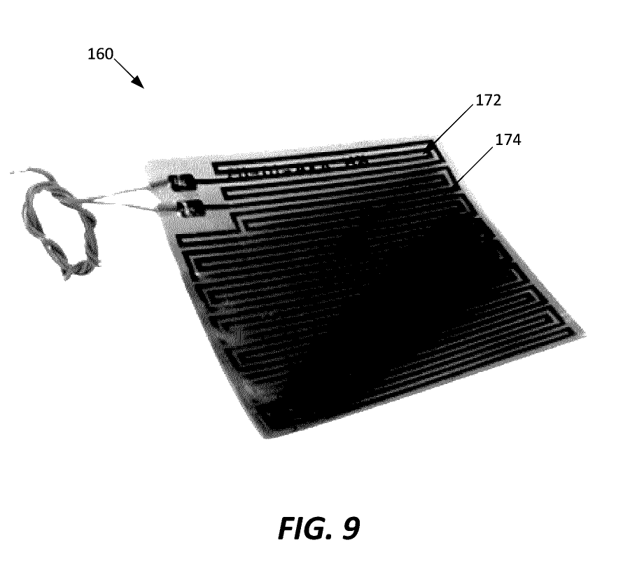

Referring to FIG. 9, the heating layer 160 includes a low-wattage resistive heating element 172. In some embodiments, the pad assembly 102 is designed for a range of about 2-5 watts for a 2″×2″ heating element. In one example, the pad assembly 102 is operable at about 0.8 amps with about 3.3 volts. In other examples, the pad assembly 102 is configured to utilize other voltages, such as in a range from about 3.3 to about 9 volts, depending on the size, shape, and/or type of the pad 140. The pad assembly 102 of the present disclosure is designed to provide sufficient wattage to deliver heat on the skin, but also operates on low voltage so that safety concerns are minimized.

In some examples, the heating layer 160 is flexible and made with resistive foils and polymers. For example, the heating layer is made a thin, flexible layer manufactured from thin plastic film such as polyester, polyimide, and other plastic films with or without adhesives formed thereon. An example of the heating element 172 includes a polyimide film with insulated flexible heating element, as illustrated in FIG. 9. In some examples, the heating layer 160 has a thickness of not greater than 0.010 inches. The heating element 172 can be rated up to 200° C. with 0.010 inches in maximum thickness. The heating element 172 is generally configured to generate heat of about 0.1 to 1.0 watts/in². This output range of the heating element 172 can meet the low-wattage characteristic of the pad assembly. In other embodiments, the heating element 172 generates about 0.05 to 5 watts/in². In yet other embodiments, the heating element 172 is configured to generate about 0.1-10 watts/in².

As illustrated in FIG. 9, the heating layer 160 can be produced using thin foils 174 as the heating element 172. Such thin foils 174 enable the heating layer 160 to be wider and flat. This allows the heating element pattern to be very close together, thereby providing even heat to the body skin. The heating element pattern can be of any design suitable for providing substantially uniform heat throughout the body skin on which the pad 140 is attached.

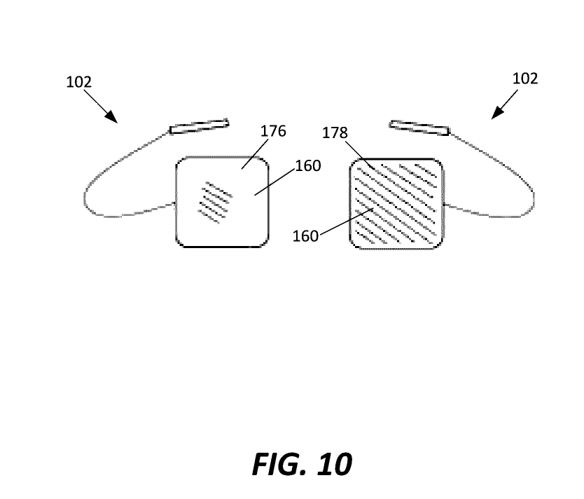

Alternatively, as illustrated in FIG. 10, there are other thin heater technologies that can also be utilized for the heating layer 160. An example of one such heater includes polymer resistive inks on plastic film. Resistive inks are printed on plastic films to provide the desired patterns or shapes intended to be applied to the body skin in a desired shape or configuration. In still further examples, carbon electrodes 176 are used for the heating layer 160. For example, a carbon-impregnated polymer material could be used, in which an extruded material can be shaped in a manner to provide a pattern analogous to a printed ink pattern. In yet other examples, silver coated electrodes 178 are used for the heating layer 160.

Additionally, a pattern of the heating element need not be consistent across all areas of a particular surface. For example, in various embodiments, the shape, configuration, and spacing of heating elements could vary to allow heating in desired body areas, or to concentrate heating at a particular intended area. Accordingly, when the pad 140 covers a particular skin area, the heating element can be shaped to introduce heating in various parts of that skin area while leaving other areas unheated. Additionally, within one heater, individual heating elements can be widened and/or narrowed to adjust heat output profiles of the skin area.

Referring again to FIGS. 7 and 8, the electrical stimulation layer 162 defines at least part of one or more electrical stimulation circuits. In some examples, the electrical stimulation layer 162 includes a plurality of electrical stimulation electrodes spaced apart from each other. An example of the electrical stimulation layer 162 is configured to provide transcutaneous electrical nerve stimulation (TENS) to relieve chronic pain and produce muscle building stimulation.

The cover layer 154 covers the other layers and components of the pad 140. In some examples, the cover layer 154 is made of nonconductive, flexible materials to protect the layers and components of the pad 140.

The pad circuit 156 is configured to connect the cable 142 with the stimulation layer 152 and receive the control signal 116 from the control device 104 so that the stimulation layer 152 is controlled based on the control signal 116. In some examples, the pad circuit 156 includes the pad identification device 106 to define the identification circuit. The pad circuit 156 further includes a storage device. The storage device of the pad circuit 156 can be used to store information 114 (e.g., the pad ID information) identifying the pad assembly 102. This pad ID information can include a unique identifier of the pad, from which pad characteristics can be determined based on a database of pad identifiers; in alternative embodiments, the pad ID information can also include information describing physical characteristics of the pad, such as pad size, construction (e.g., specific layers included), spacing/positioning of TENS electrodes, electrical requirements and rating of a heating layer, heat pattern selections, or other features of the pad. In some embodiments, the pad circuit 156 is formed on a printed circuit board and includes a microprocessor. In other embodiments, the storage device of the pad circuit 156 can store information associated with the pad assembly 102 in which the pad circuit 156 is included. Such information can include one or more physical characteristics of the pad assembly 102, as shown in pad control scheme data 230 and/or adhesive layer data 280 as described herein.

Referring to FIGS. 11A and 11B, example layouts of the pad identification device 106 are illustrated. The illustrated layouts of the pad identification device 106 can achieve a small package at low cost that can fit within the pad 140 or the adhesive layer 150. As illustrated, the pad identification device 106 can be formed on a printed circuit board and includes a microprocessor and a storage device. In some embodiments, the storage device of the pad identification device 106 stores the pad ID information 114. In other embodiments, the storage device of the pad identification device 106 can further store various pieces of information, such as one or more physical characteristics of the corresponding pad assembly 102, or information included in the pad control scheme data 230 and/or the adhesive layer data 280 as described herein.

Referring again to FIG. 7, the sensing element 158 is provided to detect one or more parameters associated with the pad 140. The sensing element 158 can operate as the sensing device 120 as described in FIG. 2. In some examples, the sensing element 158 is configured to monitor the temperature output from the heating layer 160. The sensing element 158 is connected to the pad circuit 156. The sensing element 158 can be disposed in various locations within the pad 140 for different measurement purposes. In other examples, the sensing element 158 is integrally formed with the pad circuit 156.

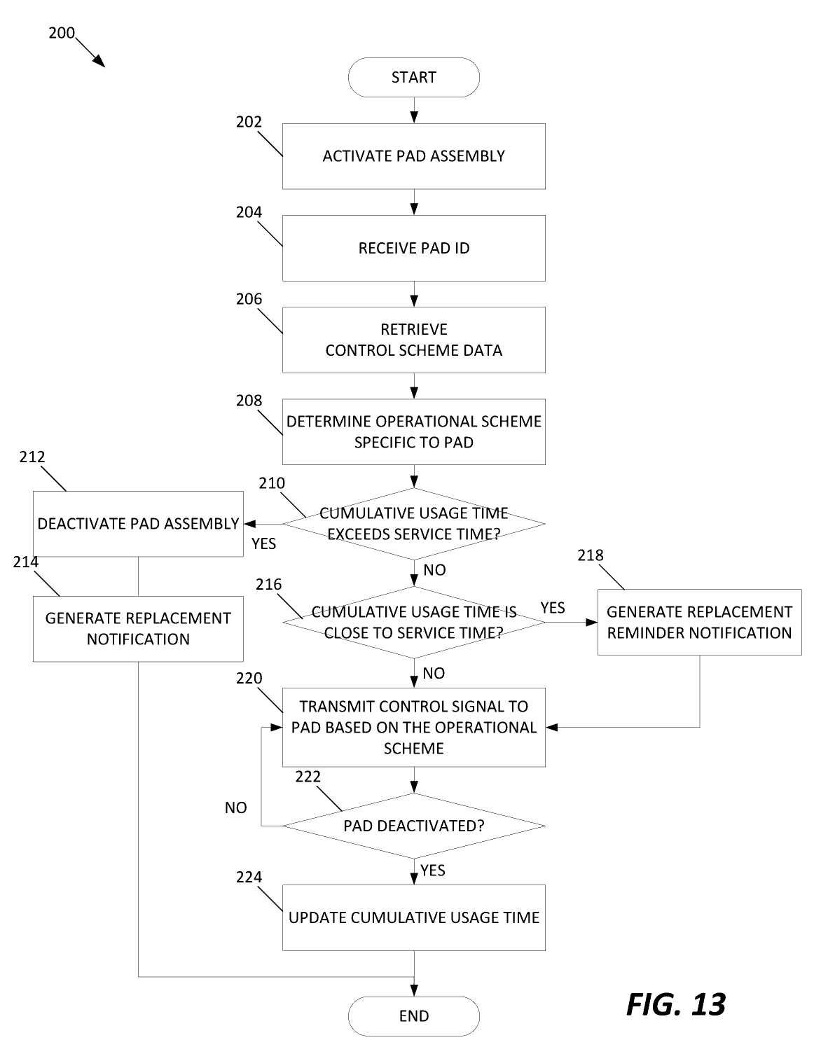

Referring now to FIGS. 13-17, various examples of operating the system 100 are described.

In general, the pad identification device 106 provides a serialization for a particular pad assembly 102, which uniquely identifies the pad assembly 102. When the pad assembly 102 is plugged into the control device 104, the control device 104 can recognize the connected pad assembly 102 from the pad ID information 114. The control device 104 and/or the pad assembly 102 (e.g., the pad identification device 106) can store data associated with a plurality of pad assemblies in the storage device thereof. The control device 104 can retrieve the information associated with the connected pad assembly 102 by matching the pad ID information 114 of the pad assembly 102 with the stored data. The retrieved information can include one or more physical characteristics, such as shape, size, and type of the pad assembly. The information can also include an operational scheme specific to the pad assembly 102.

The serialization of pad assemblies 102 enables the control device 104 to provide an appropriate amount of input power needed for each pad assembly 102. In general, different physical characteristics (e.g., size) of pad assemblies 102 requires different power input. For examples, the identification of the pad assembly 102 allows to maintain a power input (or output) per unit area consistent, based on the physical characteristics of pad assemblies. Further, the control device 104 can select a combination of thermal and electrical stimulations desirable for a particular pad assembly 102 (for example, depending on the shape, size, and/or type of the pad assembly). For example, depending on the attributes of the pad assembly 102 identified by the control device 104, the control device 104 can control the pad assembly 102 to generate only heat, or a combination of heat and electrical stimulation.

In the illustrated embodiment, the control device 104 can operate to monitor and store a cumulative time that the pad assembly 102 has been in use. Based on the cumulative time, the control device 104 can generate a notification indicating the pad assembly (e.g., the pad 140) should be replaced. Further, the control device 104 can disable the pad assembly 102 when the cumulative time exceeds a pad service time of the pad assembly 102. In addition, the control device 104 can also notify a user that the pad assembly will become unusable soon as a warning to plan to substitute the pad assembly.

Alternatively, or in addition, the control device 104 can generate a notification that the adhesive layer 150 should be replaced when the cumulative time, which the pad assembly 102 has been in used, exceeds a service time (either a service time of the pad assembly or a service time of the adhesive layer).

In these configurations, the pad identification device 106 can be integrated into either the stimulation layer 152 or the adhesive layer 150. When the pad identification device 106 is included in the adhesive layer 150 and the adhesive layer 150 is removable from the pad assembly 102 and replaceable, the pad identification device 106 can be used to provide a notification that the adhesive layer 150 should be replaced. When the adhesive layer 150 is replaced by a new adhesive layer 150 including a pad identification device 106, a new identification information for the new adhesive layer 150 can be transmitted to the control device 104 so that the control device 104 starts monitoring a cumulative time that the new adhesive layer 150 has been used.

Referring to FIG. 13, an example method 200 of operating the system 100 is described. In this embodiment, the method 200 is performed primarily by the control device 104 in connection with the pad assembly 102. In other embodiments, the method 200 can be executed at least partially by the pad assembly 102 that is connected to the control device 104.

In operation 202, the control device 104 activates the pad assembly 102 by supplying electric power to the pad assembly 102. In particular, when the pad assembly 102 is connected to the control device 104, the pad identification device 106 is activated to transmit the pad ID information 114 to the control device 104.

In operation 204, the control device 104 receives the pad ID information 114 from the pad assembly 102 connected to the control device 104.

In operation 206, the control device 104 retrieves pad control scheme data 230. As described in detail with respect to FIG. 14, the pad control scheme data 230 include information associated with a plurality of pad assemblies 102, such as physical characteristics (e.g., size, shape, and type) and operational schemes.

In operation 208, the control device 104 determines the physical characteristics and/or the operational schemes specific to the pad assembly 102 from the pad control scheme data 230.

In operation 210, the control device 104 determines whether a cumulative time that the pad assembly 102 has been in use exceeds a service time of the pad assembly 102. If it is determined that the cumulative time exceeds the pad service time (“YES” in the operation 210), the method 200 moves on to operation 212. Otherwise (“NO” in the operation 210), the method 200 continues to operation 216.

In operation 212, the control device 104 operates to deactivate the pad assembly 102 so that the pad assembly 102 is disabled until the pad assembly 102 is replaced by a new pad assembly 102.

In operation 214, the control device 104 generates a pad replacement notification based on the cumulative time exceeding the pad service time. The pad replacement notification is adapted to indicate that the pad assembly 102 should be replaced.

In operation 216, the control device 104 determines whether the cumulative time is so close to the pad service time as to generate a pad replacement reminder notification. In some embodiments, the cumulative time is determined to be close enough if the cumulative time exceeds a certain threshold. The threshold can be predetermined in various ways. For example, the threshold can be a certain ratio or percentage of the cumulative time over the pad service time (e.g., 90% of the pad service time). Alternatively, the threshold can be a certain difference between the cumulative and the pad service time (e.g., 10 hours left to the pad service time). If it is determined that the cumulative time exceeds the threshold (“YES” in the operation 216), the method 200 continues in operation 218. Otherwise (“NO” in the operation 216), the method 200 moves on to operation 220.

In operation 218, the control device 104 generates a pad replacement reminder notification to indicate that the pad assembly 102 should be replaced in the near future.

In operation 220, the control device 104 transmits the control signal 116 to the pad assembly 102 based on the operational scheme specific to the pad assembly 102, which has been determined from the pad control scheme data 230.

In operation 222, the control device 104 detects that the pad assembly 102 is deactivated or disabled. If so (“YES” in the operation 222), the method 200 moves on to operation 224. Otherwise (“NO” in the operation 222), the operation 220 continues.

In operation 224, the control device 104 updates the cumulative time to include a time the pad assembly 102 has been used over the current period of pad activation. This pad usage time is added to the cumulative time that the pad assembly 102 has been used.

Referring to FIG. 14, an example of the pad control scheme data 230 is illustrated. The pad control scheme data 230 includes information associated with a plurality of pad assemblies 102 that can be used with the control device 104. In the illustrated embodiment, the pad control scheme data 230 include physical characteristics 232, such as size, shape, and type, of each pad assembly 102, and operational schemes 234, such as input power, heat pattern, electrical stimulation pattern, service life, and cumulative usage time, of each pad assembly 102. In some embodiments, the input power indicates electric power delivered to the heating layer 160 of the pad assembly 102. The heat pattern can represent a pattern of control signal to adjust a heat profile generated by the heating layer 160. The electrical stimulation pattern includes a type or pattern of stimulation applied via the plurality of electrical stimulation electrodes of the electrical stimulation layer 162 of the pad assembly 102. The service life indicates a life that the pad assembly 102 can be reliably and safely used for intended purposes. The cumulative usage time indicates a cumulative time that the pad assembly 102 has been in use or activated.

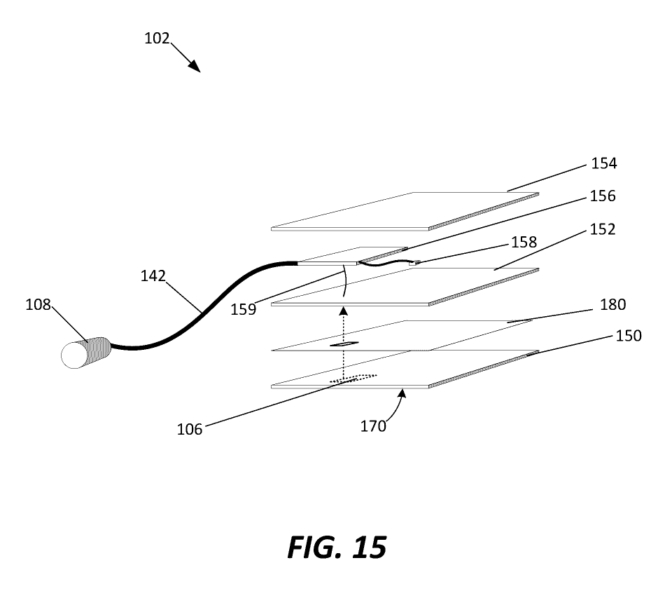

Referring to FIGS. 15-18, another example of the pad assembly 102 is described. In this example, the pad identification device 106 is incorporated with the adhesive layer 150. Other than the configuration of the pad identification device 106, the pad assembly 102 is configured similarly to the pad assembly 102 shown in FIGS. 7 and 8. Therefore, where like or similar features or elements are shown, the same reference numbers will be used where possible. The following description will be limited primarily to the differences from the pad assembly 102 of FIGS. 7-8, 13, and 14.

In this example, the adhesive layer 150 is configured to be removable while leaving the rest of the pad assembly 102, and replaceable with another adhesive layer 150. Such adhesive layers (e.g., hydrogel) can be sold and marketed independently for replacement. Further, the pad identification device 106 is incorporated in the adhesive layer 150. In some embodiments, the adhesive layer 150 is attached to the rest of the pad 140 with an additional adhesive tape or layer 180 (FIG. 15) disposed therebetween. The adhesive tape 180 has sufficiently strong bond strength to hold the pad 140 with the adhesive layer 150 together in normal use. The bond strength of the adhesive tape 180 to the pad 140 can be higher than a bond of the adhesive layer 150 to the skin. However, the bond between the adhesive tape 180 and the adhesive layer 150 is adapted to enable a user to peel off the adhesive layer 150 from the pad 140 (either with or without the adhesive tape 180). An example of the adhesive tape 180 is a model number 9425HT from 3M Company of St. Paul, Minn.

The pad identification device 106 incorporated in the adhesive layer 150 can be connected to the connector 108 in various manners. In some examples, the pad circuit 156 provides electrical contacts that are exposed when the adhesive layer 150 is removed from the pad 140. The electrical contacts of the pad circuit 156 are configured to be connected to corresponding contacts of an adhesive layer 150 when the adhesive layer 150 is mounted to the pad 140. Such electrical contacts of the pad circuit 156 can extend out from the pad circuit 156 (through a cable 159, for example) and be arranged to a position at which the pad identification device 106 can be repeatedly located when the adhesive layer 150 is attached to the pad 140. In other examples, the electrical contacts of the pad circuit 156 include electrical pins extending therefrom toward the adhesive layer 150. Depending on the arrangement of the pad circuit 156 relative to the layers and components of the pad assembly 102, the electrical contacts of the pad circuit 156 can be located differently. Alternatively, the pad identification device 106 of an adhesive layer 150 can be directly connected to the connector 108 through the cable 142 and/or another cable when the adhesive layer 150 is engaged with the pad 140. Further, the pad 150 can provide a snap-fit mechanism to engage the adhesive layer 150 with the pad 140, in addition to, or alternatively to, the adhesive tape 180.

The replacement of the adhesive layer 150 can address various issues. As heat is applied to the adhesive layer, the material (e.g., hydrogel) of the adhesive layer can deteriorate and lose its adhesion properties over time. For example, the performance (e.g., tackiness) of the adhesive layer can decrease as heat is repeatedly applied to the adhesive layer. The pad assembly of the present disclosure, in which the pad identification device is incorporated in the replaceable adhesive layer, can allow the control device to determine a replacement time for the adhesive layer and even disable the pad assembly when the adhesive layer is degraded too much. Further, the replacement of the adhesive layer, rather than a replacement of the overall pad or pad assembly, is cost efficient because the expensive parts of the pad assembly, such as wiring, heating layer, and electro-stimulation layer, can be reused without replacement.

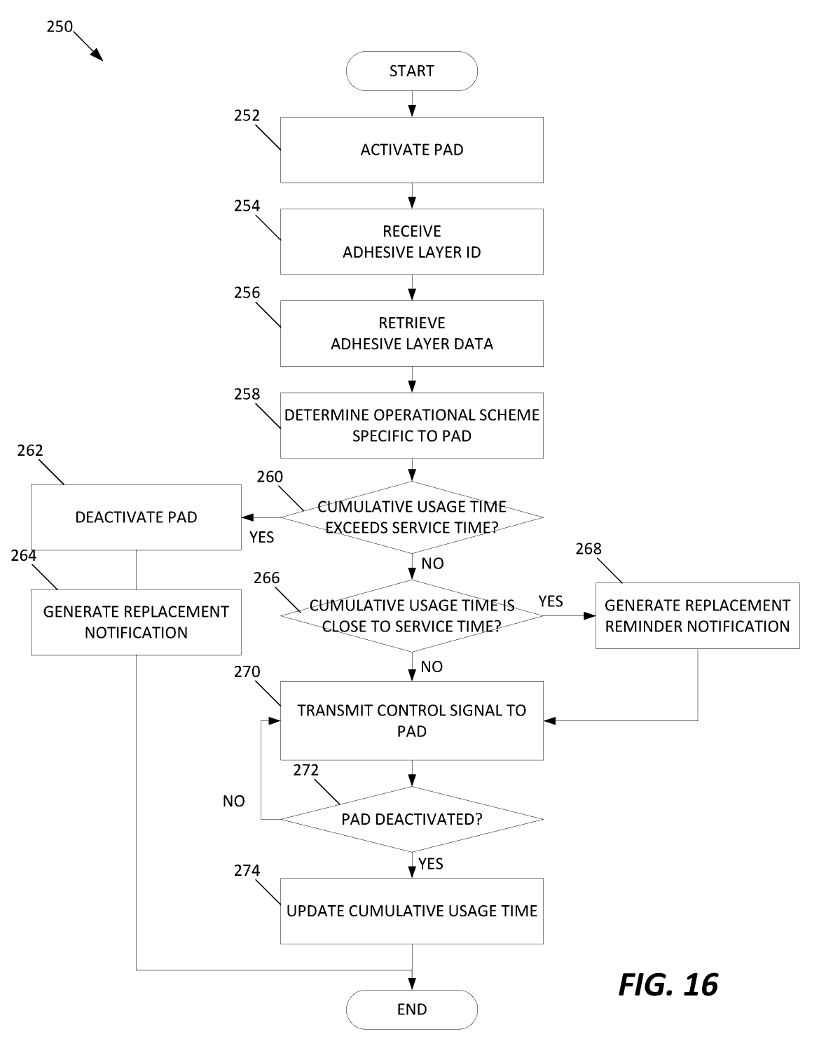

Referring to FIG. 16, another example method 250 of operating the system 100 is described. In this example, the method 250 is performed with the pad assembly 102 of FIG. 15. In this embodiment, the method 250 is performed primarily by the control device 104 in connection with the pad assembly 102. In other embodiments, the method 250 can be executed at least partially by the pad assembly 102 that is connected to the control device 104.

In operation 252, the control device 104 activates the pad assembly 102 by supplying electric power to the pad assembly 102. In particular, when the pad assembly 102 is connected to the control device 104, the pad identification device 106 is activated to transmit the pad ID information 114 to the control device 104.

In operation 254, the control device 104 receives information 286 (FIG. 17) identifying the adhesive layer 150 of the pad assembly connected to the control device 104. Such adhesive layer ID information 286 can be part of the pad ID information 114.

In operation 256, the control device 104 retrieves adhesive layer data 280 (FIG. 17). As described in detail with respect to FIG. 17, the adhesive layer data 280 include information associated with a plurality of adhesive layers 150, such as physical characteristics (e.g., size, shape, and type) and operational schemes (e.g., service time and cumulative usage time). The adhesive layer data 280 can be included in the pad control scheme data 230. For example, the physical characteristics and operational schemes of the pad control scheme data 230 are organized to include the physical characteristics and operational schemes of the adhesive layer data 280.

In operation 258, the control device 104 determines the physical characteristics and/or the operational schemes specific to the pad assembly 102 from the pad control scheme data 230, as in the operation 208 (FIG. 13).

In operation 260, the control device 104 determines whether a cumulative time that the adhesive layer 150 has been in use exceeds a service time of the adhesive layer 150. If it is determined that the cumulative time exceeds the adhesive layer service time (“YES” in the operation 260), the method 250 moves on to operation 262. Otherwise (“NO” in the operation 260), the method 250 continues to operation 266.

In operation 262, the control device 104 operates to deactivate the pad assembly 102 so that the pad assembly 102 is disabled until the adhesive layer 150 is replaced by a new adhesive layer 150.

In operation 264, the control device 104 generates an adhesive layer replacement notification based on the cumulative time exceeding the adhesive layer service time. The adhesive layer replacement notification is adapted to indicate that the adhesive layer 150 should be replaced.

In operation 266, the control device 104 determines whether the cumulative time is so close to the adhesive layer service time as to generate a adhesive layer replacement reminder notification. In some embodiments, the cumulative time is determined to be close enough if the cumulative time exceeds a certain threshold. The threshold can be predetermined in various ways. For example, the threshold can be a certain ratio or percentage of the cumulative time over the adhesive layer service time (e.g., 90% of the adhesive layer service time). Alternatively, the threshold can be a certain difference between the cumulative and the adhesive layer service time (e.g., 10 hours left to the adhesive layer service time). If it is determined that the cumulative time exceeds the threshold (“YES” in the operation 266), the method 250 continues in operation 268. Otherwise (“NO” in the operation 266), the method 250 moves on to operation 220.

In operation 268, the control device 104 generates an adhesive layer replacement reminder notification to indicate that the adhesive layer 150 should be replaced in the near future.

In operation 270, the control device 104 transmits the control signal 116 to the pad assembly 102 based on the operational scheme specific to the pad assembly 102, which has been determined from the pad control scheme data 230, as in the operation 220 (FIG. 13).

In operation 272, the control device 104 detects that the pad assembly 102 is deactivated or disabled. If so (“YES” in the operation 272), the method 250 moves on to operation 274. Otherwise (“NO” in the operation 272), the operation 270 continues.

In operation 274, the control device 104 updates the cumulative time to include a time the adhesive layer 150 has been used over the current period of pad activation. This adhesive layer usage time is added to the cumulative time that the adhesive layer 150 has been used.

Referring to FIG. 17, an example of the adhesive layer data 280 is illustrated. The adhesive layer data 280 include information associated with a plurality of adhesive layers 150 that can be affixed to the pad 140. In the illustrated embodiment, the adhesive layer data 280 include physical characteristics 282, such as size, shape, and type, of each adhesive layer 150, and operational schemes 284, such as service life and cumulative usage time, of each adhesive layer 150. In some embodiments, the service life indicates a life that the adhesive layer 150 can be reliably and safely used for intended purposes. The cumulative usage time indicates a cumulative time that the adhesive layer 150 has been in use with the pad 140.

Referring again to FIGS. 8 and 9, the heating layer 160 of the pad 140 is controlled by the control device 104 via the pad circuit 156 to provide various thermal profiles. For example, the control device 104 controls the input voltage (e.g., as part of the control signal 116) to the heating layer 160 to provide different heating patterns, durations, and/or exposure times.

In some embodiments, the overall heat output of the heating layer 160 is controlled by closely matching the input voltage to the heating layer 160, thereby resulting in precise wattage. In other embodiments, the overall heat output of the heating layer 160 is controlled by varying input voltage and/or current. This variation of input voltage and/or current can produce heat rise and decline cycles with various heat intensity levels of different patterns.

The control device 104 is also configured to introduce interruptions in the input power to the heating layer 160 to control the heat output of the heating layer 160. In some embodiments, such interruptions are of varying time durations.

As described herein, the pad 140 is shaped in such a pattern as to provide improved contact with the skin while maintaining flexibility on different surface topology of the skin. Some examples of different shapes and/or sizes of the pad 140 are illustrated in FIG. 12.

Referring again to FIG. 8, the pad assembly 102 can include the programmable heating layer 160 fabricated in combination with the electrical stimulation layer 162 (e.g., TENS layer) so that a single patch is attached to a skin to deliver both electrical stimulation patterning and programmable thermal patterning to the skin. In some embodiments, a single pad assembly can include either one or two cables attached thereto such that the heating layer and the electrical stimulation layer can be attached to a skin for simultaneous thermal and electrical therapeutic treatments. The control device 104 can control the combination of the heating and electrical stimulation layers collectively or independently. This configuration allows generating a large variety of combinations of electrical stimulation patterns and thermal variations. The combination of variable thermal and electrical stimulation patterns can produce enhanced therapeutic results and pain management.

In some embodiments, the heating layer 160 and the electrical stimulation layer 162 (e.g., polymer carbon inks) can be laminated and sandwiched into a single layer. As described herein, such a laminated electrical stimulation and heating layer can be attached to a skin using a skin friendly adhesive layer 150, such as a hydrogel layer.

The system 100 in accordance with the present disclosure provides a control mechanism that delivers both electrical stimulation variations and heating variations from a single control device. Further, the pad assembly 102 with the combination of the heating and electrical stimulation layers can be operated with low voltage, such as lower than 12 volts, so as to avoid damage to the skin that is exposed to the electric current. Further, the control device 104 can operate to automatically limit an amount of time that the heating layer 160 is in use at a time to apply heat, thereby avoiding damage to the skin that may result from prolonged heat exposure at elevated levels.

In some embodiments, the control device 104 is configured to control the heating layer 160 to generate a pattern of heat in such a cycle that the output temperature of the heating layer 160 alternatingly increases above a predetermined temperature and decrease below the predetermined temperature. One example of the predetermined temperature is about 104° F. This heating pattern can allow the skin to recover from the higher temperature and thus be subject to higher heat exposure levels alternatingly. Accordingly, the user can have prolonged safe exposure to the heat without skin damage and thus take advantage of increased pain relief and therapeutic treatment.

As described herein, the pad 140 with a combination of the heating layer and the electrical stimulation layer is shaped in such a way as to provide highly localized heat application on the subject's body without affecting nearby areas. For example, the pad 140 can be applied only on the knuckles of a hand to assist with arthritic problems.

As shown in FIGS. 2, 7, and 15, the sensing device is integrated into the pad assembly 102 and configured to detect the output temperature at the interface between the pad and the skin. The detected temperature can be used as a feedback by the control device 104 for improved controlling of the pad assembly.

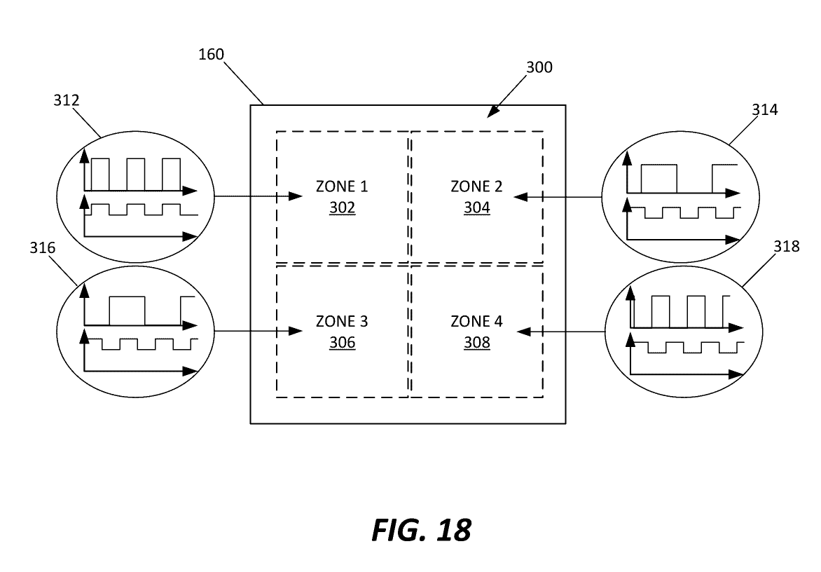

Referring to FIG. 18, the heating layer 160 can define a plurality of heating zones 300. In the illustrated example, the heating layer 160 includes four heating zones (i.e., first, second, third, and fourth heating zones 302, 304, 306, and 308. The heating zones 300 can be independently controlled by the control device 104 based on different operational schemes. Such operational schemes for different heating zones of each pad assembly can be defined in the pad control scheme data 230 for that pad assembly. The multiple heating zones within a single pad can provide advanced thermal delivery and profiles for different skin contact regions corresponding to the heating zones. In the illustrated example, the first, second, third, and fourth heating zones 302, 304, 306, and 308 are controlled with different input profiles (e.g. input voltages) 312, 314, 316, and 318, respectively. The input profiles depicted in FIG. 18 are only for illustrative purposes. In other examples, different heating zones have different resistances to generate different levels of heating with a common input voltage. In yet other examples, different heating zones are connected in series so that heating zones closer to the input line of the series generate higher temperatures while heating zones farther from the input line (i.e., closer to the output line) produce lower temperatures.

In some embodiments, the pad assembly 102 of the present disclosure employs a quick plug connect/disconnect mechanism.

The control device 104 can be configured to control a plurality of pad assemblies independently. The quick plug connect/disconnect mechanism of the pad assembly 102 can facilitate the connection of the plurality of pad assemblies to the control device. In some embodiments, the control device 104 can include a control logic that first detects presence or connection of one or more pad assemblies 102. In some embodiments, the control device 104 can monitor the resistance associated with the pad assemblies 102 to detect if one or more pad assemblies are connected to the control device 104 for a therapy session. Based on the connection status, the control device 104 can adjust the control scheme for one or more of the pad assemblies connected thereto. Each of the pad assemblies can then be controlled independently in the same or similar manner as described above. For example, the control device 104 can operate to supply different amounts of input power to different pad assemblies and adjust how the sensing devices are interpreted for different pad assemblies. Further, the control device 104 can identify each of the pad assemblies connected thereto based on the pad ID information 114 from each pad assembly. As described herein, each of the pad assemblies or each of the adhesive layers can be monitored for its cumulative usage time, which can be used to notify a user of the decline in efficiency and performance (e.g., heat delivery or loss of tackiness). Each of the pad assemblies can be disabled if the pad or the adhesive layer reaches a preset level, such as the service life. The feature of monitoring the cumulative usage time can also be used to provide a manufacturer with the ability to preset the action that is to take place based on the monitored usage hours.

The control device 104 of the present disclosure is configured to provide thermal patterning through the pad assembly 102 that improves the service life and performance of the pad or the adhesive layer (e.g., hydrogel).

In some embodiments, the control device 104 is configured to monitor the resistance of the pad assembly 102 (e.g., the heating layer 160), and determine a heater wattage capability of the pad assembly 102. The control device 104 then operates to adjust the voltage to the pad assembly 102 (e.g., the heating layer 160) based on the heater wattage capability, and deliver proper thermal output through the pad assembly 102.

In some embodiments, the system 100 in the present disclosure can provide an open-loop control system in which the wattage output of the pad assembly 102 (e.g., the heating layer thereof) is controlled by the input voltage, thereby eliminating the need for a control system based upon sensing the output of the pad assembly 102. In this configuration, the wattage level can be determined solely with a consistent input voltage that produces the desired temperature of the pad 140.

The system 100 of the present disclosure is suitable for any location that pain relieving, relaxing, or similarly pleasant effect is required on the subject's body or on body parts by means of heat and/or electrical stimulation. Examples of such treatment are rheumatic complaints, muscle tension, back complaints, chills of the jawbone and sinuses, bladder, kidneys, menstrual complaints, nerve pain, whiplash syndrome or reinforcement of the absorption of remedies which are applied to body parts in the form of lotions. The pad assembly 102 can also be used for keeping warm or heating up exposed body parts, such as hands and feet.

FIG. 19 illustrates an exemplary architecture of the control device 104. The control device 104 can include at least some of the elements illustrated in FIG. 19. The control device 104 illustrated in FIG. 19 is used to execute the operating system, application programs, and software modules (including the software engines) described herein.

The control device 104 is a computing device of various types. In some embodiments, the control device 104 is a mobile computing device. Examples of the control device 104 as a mobile computing device include a mobile device (e.g., a smart phone and a tablet computer), a wearable computer (e.g., a smartwatch and a head-mounted display), a personal digital assistant (PDA), a handheld game console, a portable media player, a ultra-mobile PC, a digital still camera, a digital video camera, and other mobile devices. In other embodiments, the control device 104 is other computing devices, such as a desktop computer, a laptop computer, or other devices configured to process digital instructions.

It is recognized that the architecture illustrated in FIG. 19 can also be implemented in other computing devices used to achieve aspects of the present disclosure. To avoid undue repetition, this description of the control device 104 will not be separately repeated herein for each of the other computing devices.

The control device 104 includes, in some embodiments, at least one processing device 402, such as a central processing unit (CPU). A variety of processing devices are available from a variety of manufacturers, for example, Intel or Advanced Micro Devices. In this example, the control device 104 also includes a system memory 404, and a system bus 406 that couples various system components including the system memory 404 to the processing device 402. The system bus 406 is one of any number of types of bus structures including a memory bus, or memory controller; a peripheral bus; and a local bus using any of a variety of bus architectures.

The system memory 404 includes read only memory 408 and random access memory 410. A basic input/output system 412 containing the basic routines that act to transfer information within the control device 104, such as during start up, is typically stored in the read only memory 408.

The control device 104 also includes a secondary storage device 414 in some embodiments, such as a hard disk drive, for storing digital data. The secondary storage device 414 is connected to the system bus 406 by a secondary storage interface 416. The secondary storage devices and their associated computer readable media provide nonvolatile storage of computer readable instructions (including application programs and program modules), data structures, and other data for the control device 104.

Although the exemplary environment described herein employs a hard disk drive as a secondary storage device, other types of computer readable storage media are used in other embodiments. Examples of these other types of computer readable storage media include magnetic cassettes, flash memory cards, digital video disks, Bernoulli cartridges, compact disc read only memories, digital versatile disk read only memories, random access memories, or read only memories. Some embodiments include non-transitory media.

A number of program modules can be stored in secondary storage device 414 or memory 404, including an operating system 418, one or more application programs 420, other program modules 422, and program data 424.

In some embodiments, the control device 104 includes input devices to enable a user to provide inputs to the control device 104. Examples of input devices 426 include a keyboard 428, a pointer input device 430, a microphone 432, and a touch sensitive display 440. Other embodiments include other input devices. The input devices are often connected to the processing device 402 through an input/output interface 438 that is coupled to the system bus 406. These input devices 426 can be connected by any number of input/output interfaces, such as a parallel port, serial port, game port, or a universal serial bus. Wireless communication between input devices and interface 438 is possible as well, and includes infrared, BLUETOOTH® wireless technology, 802.11a/b/g/n, cellular, or other radio frequency communication systems in some possible embodiments.

In this example embodiment, a touch sensitive display device 440 is also connected to the system bus 406 via an interface, such as a video adapter 442. The touch sensitive display device 440 includes touch sensors for receiving input from a user when the user touches the display. Such sensors can be capacitive sensors, pressure sensors, or other touch sensors. The sensors not only detect contact with the display, but also the location of the contact and movement of the contact over time. For example, a user can move a finger or stylus across the screen to provide written inputs. The written inputs are evaluated and, in some embodiments, converted into text inputs.

In addition to the display device 440, the control device 104 can include various other peripheral devices (not shown), such as speakers or a printer.

When used in a local area networking environment or a wide area networking environment (such as the Internet), the control device 104 is typically connected to the network through a network interface, such as a wireless network interface 446. Other possible embodiments use other communication devices. For example, some embodiments of the control device 104 include an Ethernet network interface, or a modem for communicating across the network.

The control device 104 typically includes at least some form of computer-readable media. Computer readable media includes any available media that can be accessed by the control device 104. By way of example, computer-readable media include computer readable storage media and computer readable communication media.

Computer readable storage media includes volatile and nonvolatile, removable and non-removable media implemented in any device configured to store information such as computer readable instructions, data structures, program modules or other data. Computer readable storage media includes, but is not limited to, random access memory, read only memory, electrically erasable programmable read only memory, flash memory or other memory technology, compact disc read only memory, digital versatile disks or other optical storage, magnetic cassettes, magnetic tape, magnetic disk storage or other magnetic storage devices, or any other medium that can be used to store the desired information and that can be accessed by the control device 104. Computer readable storage media does not include computer readable communication media.

Computer readable communication media typically embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are also included within the scope of computer readable media.

The computing device illustrated in FIG. 19 is also an example of programmable electronics, which may include one or more such computing devices, and when multiple computing devices are included, such computing devices can be coupled together with a suitable data communication network so as to collectively perform the various functions, methods, or operations disclosed herein.

Referring again to FIG. 19, the control device 104 can include a location identification device 448. The location identification device 448 is configured to identify the location or geolocation of the control device 104. The location identification device 448 can use various types of geolocating or positioning systems, such as network-based systems, handset-based systems, SIM-based systems, Wi-Fi positioning systems, and hybrid positioning systems. Network-based systems utilize service provider's network infrastructure, such as cell tower triangulation. Handset-based systems typically use the Global Positioning System (GPS). Wi-Fi positioning systems can be used when GPS is inadequate due to various causes including multipath and signal blockage indoors. Hybrid positioning systems use a combination of network-based and handset-based technologies for location determination, such as Assisted GPS.

Referring again to FIG. 19, the control device 104 further includes a short-range wireless communication device 450. The short-range wireless communication device 450 is configured to establish short-range wireless communication with the pad assembly 102. Short-range wireless communication is one-way or two-way short-range to medium-range wireless communication. Short-range wireless communication can be established according to various technologies and protocols. Examples of short-range wireless communication include a radio frequency identification (RFID), a near field communication (NFC), a Bluetooth technology, and a Wi-Fi technology.

The various examples and teachings described above are provided by way of illustration only and should not be construed to limit the scope of the present disclosure. Those skilled in the art will readily recognize various modifications and changes that may be made without following the example examples and applications illustrated and described herein, and without departing from the true spirit and scope of the present disclosure.

1. A system for electrically generating therapeutic stimulation, the system comprising:
at least one pad assembly, each including:
a skin attachment surface including an adhesive formed thereon, the adhesive providing detachable, reusable adhesion to a skin area of a patient;
a heating layer within the pad assembly, the heating layer including a low-wattage resistive heating element; and
a pad identification circuit including a storage device, the storage device storing information identifying the pad assembly; and
a control device communicatively connected to the pad identification circuit and electrically connected to the heating layer, the control device configured to identify the pad assembly based on the information and determine an operational scheme specific to the pad assembly.

2. The system of claim 1, wherein the pad assembly further includes one or more electrical stimulation circuits including a plurality of electrical stimulation electrodes spaced apart from each other.

3. The system of claim 2, wherein the operational scheme includes at least one of electric power delivered to the heating layer of the pad assembly and a stimulation pattern applied via the plurality of electrical stimulation electrodes.

4. The system of claim 1, wherein the control device is configured to monitor a first cumulative time that the pad has been in use and generate a pad replacement notification based on the first cumulative time exceeding a predetermined pad service time, the pad replacement notification indicating that the pad assembly should be replaced.

5. The system of claim 4, wherein the control device is further configured to generate a pad replacement reminder notification when the first cumulative time exceeds a first threshold.

6. The system of claim 1, wherein the skin attachment surface is removable from the pad assembly and replaceable.

7. The system of claim 6, wherein the physical characteristics of the pad assembly include physical characteristics of the skin attachment surface.

8. The system of claim 7, wherein the control device is configured to monitor a second cumulative time that the skin attachment surface has been used and generate a layer replacement notification when the second cumulative time exceeds a layer service time, the layer replacement notification informing that the skin attachment surface needs to be replaced.

9. The system of claim 8, wherein the control device is further configured to generate a layer replacement reminder notification when the second cumulative time exceeds a second threshold.

10. The system of claim 6, wherein the pad identification device is incorporated in the skin attachment surface.

11. The system of claim 1, wherein the heating layer includes a plurality of heating zones controlled independently based on the operational scheme.

12. The system of claim 2, wherein the plurality of electrical stimulation electrodes defines a plurality of electrical stimulation zones controlled independently based on the operational scheme.

13. The system of claim 1, wherein the storage device further stores information describing one or more physical characteristics of the pad assembly.

14. A pad assembly for electrically generating therapeutic stimulation, the pad assembly comprising:
a skin attachment surface including an adhesive formed thereon, the adhesive providing detachable, reusable adhesion to a skin area of a patient;
a heating layer within the pad assembly, the heating layer including a low-wattage resistive heating element; and
a pad identification circuit including a storage device, the storage device storing information identifying the pad assembly, the pad identification circuit configured to be communicatively connected to a control device and transmit the information to the control device to enable the control device to identify the pad assembly and determine an operational scheme specific to the pad assembly.

15. The pad assembly of claim 14, wherein the heating layer includes a plurality of heating zones controlled independently based on the operational scheme.

16. The pad assembly of claim 14, wherein the pad assembly further includes one or more electrical stimulation circuits including a plurality of electrical stimulation electrodes spaced apart from each other and attachable to the skin area of the patient.

17. The pad assembly of claim 14, wherein the operational scheme includes at least one of electric power delivered to the heating layer of the pad assembly and a stimulation pattern applied via the plurality of electrical stimulation electrodes.

18. The pad assembly of claim 14, wherein the skin attachment surface is removable from the pad assembly and replaceable.

19. The pad assembly of claim 18, wherein the physical characteristics of the pad assembly include physical characteristics of the skin attachment surface, and wherein the pad identification device is incorporated in the skin attachment surface.

20. The system of claim 15, wherein the plurality of electrical stimulation electrodes defines a plurality of electrical stimulation zones controlled independently based on the operational scheme.
